(19) United States
(12) Patent Application Publication
(10) Pub. No.: US 2026/0083868 A1
(43) Pub. Date: Mar. 26, 2026

(54) GERMICIDAL IRRADIATION SOURCE INTEGRATION WITH IMPLANTABLE MEDICAL DEVICES

(71) Applicant: Trishul Kapoor, Mountain Top, PA (US)

(72) Inventor: Trishul Kapoor, Mountain Top, PA (US)

(21) Appl. No.: 18/892,539

(22) Filed: Sep. 23, 2024

(51) Int. Cl. A61L 2/00; A61L 2/08; A61L 2/10

(52) U.S. Cl. CPC A61L 2103/05; A61L 2/084; A61L 2/10

(57) ABSTRACT

In one aspect, an implantable medical device comprising: a germicidal irradiation source comprising an ultraviolet germicidal irradiation (UVGI) light source, wherein the germicidal irradiation source is on an exterior surface of a portion of implantable medical device, and wherein the UVGI light source is configured to emits a UVGI light to kill or inactivate microorganisms; an implantable power generator comprising a power source configured to provide power to the implantable medical device and the germicidal irradiation source.

### BACKGROUND

According to the Agency for Healthcare Research and Quality (AHRQ), over 10 million patients undergo surgical procedures with inpatient hospitalizations each year. With any surgical procedure, one of the most feared, and preventable, surgical complications are surgical site infections (SSI). SSI is defined by the Center for Disease Control and Prevention (CDC) as any infection related to an operative procedure at or near the surgical incision within 30 days of the procedure. SSIs occur in 2% to 4% of all account for 2-4% of all patients undergoing surgical procedures. They are the leading cause of readmissions and approximately 3% of patients with a SSI will die as a consequence. An episode of SSI can result in hospital associated costs ranging from $5000-$20,000 per episode. The estimated annual healthcare cost associated with SSIs is $3.5 billion to $10 billion. The CDC and World Health Organization (WHO) have developed several guidelines in efforts to prevent SSI. These guidelines focus around developing surgical safety checklists and promoting antibiotic prophylaxis preoperatively. There are more than 50,000 spinal cord stimulators, 100,000 implantable cardioverter defibrillators (ICDs), and 250,000 pacemakers implanted every year.

### BRIEF SUMMARY OF THE INVENTION

In one aspect, an implantable medical device comprising: a germicidal irradiation source comprising an ultraviolet germicidal irradiation (UVGI) light source, wherein the germicidal irradiation source is on an exterior surface of a portion of implantable medical device, and wherein the UVGI light source is configured to emits a UVGI light to kill or inactivate microorganisms; an implantable power generator comprising a power source configured to provide power to the implantable medical device and the germicidal irradiation source.

### BRIEF DESCRIPTION OF THE DRAWINGS

FIG. 1 illustrates an example implantable medical device(s), according to some embodiments.

FIG. 2 illustrates an example process for implementing an implantable germicidal irradiation source(s), according to some embodiments.

FIG. 3 illustrates an example implantable pulse generator with a light-source embedding in outer frame, according to some embodiments.

The Figures described above are a representative set and are not an exhaustive with respect to embodying the invention.

### DESCRIPTION

Disclosed are a system, method, and article of manufacture for germicidal irradiation source integration with implantable medical devices. The following description is presented to enable a person of ordinary skill in the art to make and use the various embodiments. Descriptions of specific devices, techniques, and applications are provided only as examples. Various modifications to the examples described herein can be readily apparent to those of ordinary skill in the art, and the general principles defined herein may be applied to other examples and applications without departing from the spirit and scope of the various embodiments.

Reference throughout this specification to ‘one embodiment,’ ‘an embodiment,’ ‘one example,’ or similar language means that a particular feature, structure, or characteristic described in connection with the embodiment is included in at least one embodiment, according to some embodiments. Thus, appearances of the phrases ‘in one embodiment,’ ‘in an embodiment,’ and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art can recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, and they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

### DEFINITIONS

Artificial cardiac pacemaker, commonly referred to as simply a pacemaker, is an implanted medical device that generates electrical pulses delivered by electrodes to one or more of the chambers of the heart. Each pulse causes the targeted chamber(s) to contract and pump blood, thus regulating the function of the electrical conduction system of the heart.

Cochlear implant (CI) is a surgically implanted neuroprosthesis that provides a person who has moderate-to-profound sensorineural hearing loss with sound perception.

Implanted pulse generator (IPG) (e.g. neurostimulator) can be a battery-powered device designed to deliver electrical stimulation to the brain and/or other aspect of the nervous system. In some examples, an IPG is a battery-powered micro-electronic device that is implanted in the body and provide electrical stimulation to the nervous system. An IPG can be used for various medical functions (e.g. regulating heart rhythms, managing chronic pain, etc.). An IPG can include electric circuits and a battery. Leads (e.g. wires) are implanted and provide the electrical stimulation to a specified portion of the nervous system. For example, leads inside veins to carry electrical pulses to the heart, helping it to beat in a specific pattern/frequency.

Intrathecal administration is a route of administration for drugs via an injection into the spinal canal, or into the subarachnoid space so that it reaches the cerebrospinal fluid (CSF). It is useful in several applications, such as for spinal anesthesia, chemotherapy, or pain management. This route is also used to introduce drugs that fight certain infections, particularly post-neurosurgical.

Irradition (e.g. any light source, UVC light, UVB lights, X-rays, Infrared, etc.).

Lithium-ion (e.g. Li-ion) battery is a type of rechargeable battery that uses the reversible intercalation of Li+ ions into electronically conducting solids to store energy. In comparison with other commercial rechargeable batteries, Li-ion batteries are characterized by higher specific energy, higher energy density, higher energy efficiency, a longer cycle life, and a longer calendar life.

Mercury-vapor lamp is a gas-discharge lamp that uses an electric arc through vaporized mercury to produce light. The arc discharge can be confined to a small, fused quartz arc tube mounted within a larger soda lime or borosilicate glass bulb.

Pulsed electromagnetic field therapy (PEMFT) can include low field magnetic stimulation (LFMS). PEMFT can use electromagnetic fields to heal non-union fractures and depression.

Sodium-vapor lamp is a gas-discharge lamp that uses sodium in an excited state to produce light at a characteristic wavelength near 589 nm.

Ultraviolet germicidal irradiation (UVGI) is a disinfection technique employing ultraviolet (UV) light, particularly UV-C (180-280 nm), to kill or inactivate microorganisms. UVGI primarily inactivates microbes by damaging their genetic material, thereby inhibiting their capacity to carry out vital functions.

Example definitions for some embodiments are now provided. These example definitions can be integrated into respective example embodiments discussed infra.

These example definitions can be integrated into example embodiments of the systems and methods discussed herein.

### EXAMPLE SYSTEMS AND METHODS

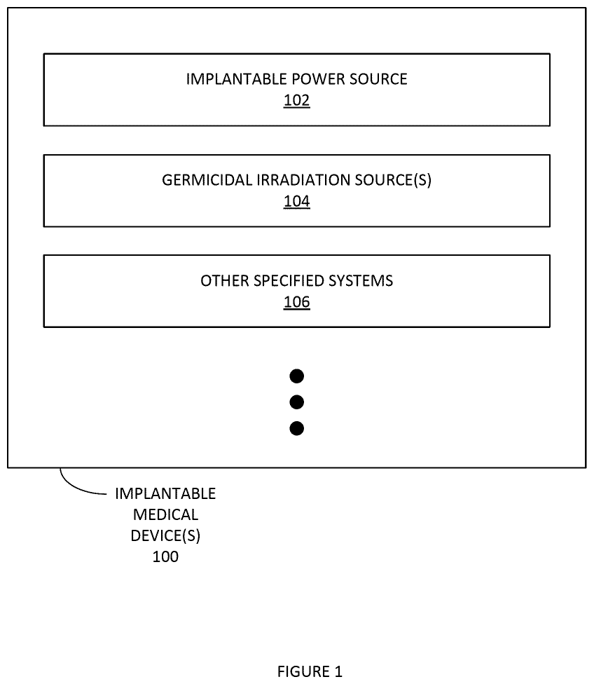

FIG. 1 illustrates an example implantable medical device(s) 100, according to some embodiments. In some examples, implantable medical device 100 can apply UVC-light production to implantable pulse generators, defibrillators, pacemakers, cochlear implants, ventricular assist devices, and artificial hearts through an external mesh casing or design a universal integration to incorporate this technology into existing manufacturing processes. UVC radiation doses can be expressed in units of joules/cm². Irradiance can be expressed in units of watts/cm² and UVC radiation is equal to irradiance multiplied by exposure time. Implantable medical device 100 can implement treatment algorithm that provided therapeutic results.

Implantable medical device 100 can include an implantable power source 102. Implantable power source 102 can be an hermetically sealed device containing a power source (e.g. a lithium battery, etc.). The lithium battery version of Implantable power source 102 can be a lithium-ion (e.g. Li-ion) battery. It is noted in some embodiments, the lithium battery version of implantable power source 102 can use lithium metal anodes with cathode systems. These cathodes can include, by way of example: iodine, carbon monofluoride, manganese oxide, silver vanadium oxide and hybrid cathodes. Lithium battery as implantable power source 102 can be charged while remaining implanted.

Other types of implantable medical device(s) 100 can include other types of system designed for each particular system. Examples of other specified systems include, inter alia: neurostimulators (e.g. deep brain stimulator, spinal cord stimulator, dorsal root ganglion stimulator, sacral nerve stimulator), drug delivery systems (e.g. intrathecal), implantable Cardiac Defibrillators, cardiac resynchronization devices, cochlear implants, bone growth generators, another type of neuroprosthesis, etc.

The implantable medical device 100 can include germicidal irradiation source(s) 104. Germicidal irradiation source(s) 104 can include, in some examples, an ultraviolet germicidal irradiation (UVGI) light source. UVGI light sources can provide a disinfection technique employing ultraviolet (UV) light. This can be, in some examples, UV-C (e.g. 180-280 nm), to kill or inactivate microorganisms. UVGI primarily inactivates microbes by damaging their genetic material, thereby inhibiting their capacity to carry out vital functions. The use of UVGI extends to an array of applications, encompassing food, surface, air, and water. disinfection. UVGI devices can inactivate microorganisms including bacteria, viruses, fungi, molds, and other pathogens.

Germicidal irradiation source(s) 104 can be used with an implantable medical device to help diminish chances of infections. In some examples, germicidal irradiation source 104 can include an electric light that produces ultraviolet C (UVC) light. This short-wave ultraviolet light disrupts DNA base pairing, causing formation of pyrimidine dimers, and leads to the inactivation of bacteria, viruses, and protozoans.

Germicidal irradiation source(s) 104 can be integrated into and/or attached to implantable medical device 100 in various ways. By way of example, germicidal irradiation source(s) 104 can be an adhesive light source to implantable medical device 100 (e.g. IPG, etc.) with induction power-source or independent battery source. By way of example, germicidal irradiation source(s) 104 can be a pocket light source for implantable medical device 100 with induction power-source or independent battery source. By way of example, germicidal irradiation source(s) 104 can be integrated by manufactured integration of light source.

Other types of Germicidal irradiation source(s) 104 can include, inter alia:. Incandescent sources, fluorescent light sources, light-emitting diode (LED) light sources, compact Fluorescent (CFL) light sources, halogen light sources, Sodium Vapor light sources, Mercury Vapor light sources, Smart Light Bulbs, etc.

Implantable medical device 100 can include an implantable power generator 104.

In some examples, the implantable medical device 100 can include other specified systems 108. In the example of a pacemaker, a sensing amplifier can be utilized. The sensing amplifier can process the electrical manifestation of naturally occurring heart beats as sensed by the heart electrodes, the computer logic for the pacemaker and the output circuitry which delivers the pacing impulse to the electrodes.

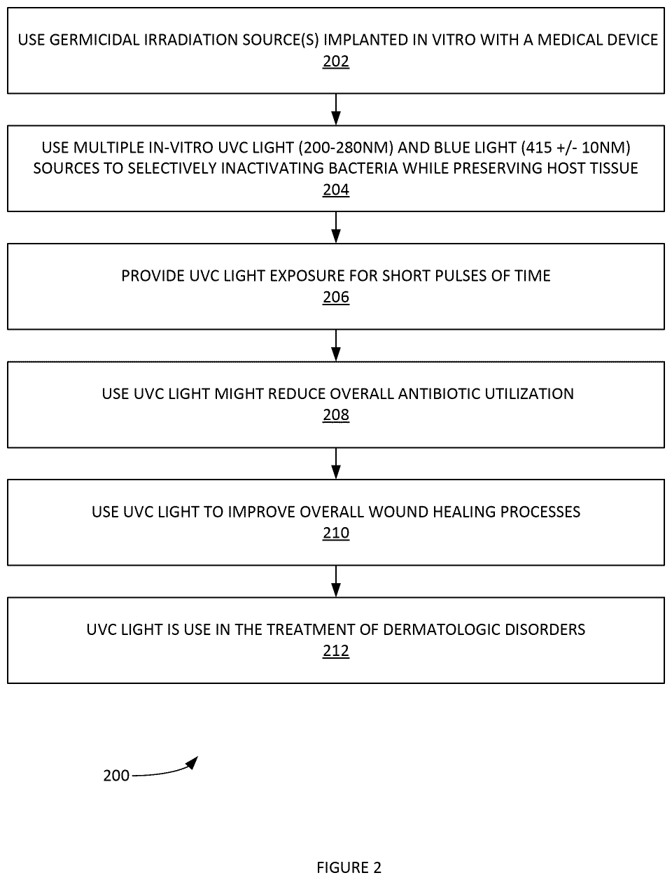

FIG. 2 illustrates an example process 200 for implementing an implantable germicidal irradiation source(s), according to some embodiments. Process 200 can use germicidal irradiation source(s) 104 implanted in vitro with a medical device in step 202. In step 204, process 200 can use multiple in-vitro UVC light (200-280 nm) and blue light (415 +/−10 nm) sources to selectively inactivating bacteria while preserving host tissue. The mechanism of action is derived from photoexcitation of endogenous porphyrins and subsequent generation of reactive oxygen species that are toxic to bacterial cells. Any potential human health hazard has been largely addressed over several studies by isolating a specific and narrow wavelength range in the far-UVC region (200-280 nm) that effectively kills bacteria but produces minimal cytotoxic, or mutagenic, damage to human cells.

In step 206, process 200 can provide UVC light exposure for short pulses of time. This can be utilized for selective inactivation of Pseudomonas Aeruginosa, Staphylococcus Aureus, and Candida Albicans while having minimal to no effect on mammalian keratinocytes. In step 208, process 200 can use UVC light might reduce overall antibiotic utilization. Accordingly, process 200 can be use with patients with poor underlying impaired perfusion as found in patients with burns and chronic wounds.

Process 200 can use UVC light to improve overall wound healing processes in step 210. Wounds heal through the process of inflammation, regeneration, and maturation. Since UVC light penetrates the skin superficially, it produces erythema, effectively promoting TGF-B and bFGF in granulation tissues to accelerate wound healing. UVC light also produces fibronectin release, epidermal hyperplasia, and increased blood flow in the microcirculation. UVC light is use in the treatment of dermatologic disorders including, inter alia: psoriasis, vitiligo, atopic dermatitis, alopecia areata, and leukoderma, etc. in step 212. UVC has several advantages in terms of low likelihood of resistance and promotion of wound healing through direct hyperplasia with enhanced re-epithelialization.

Process 200 (and system 100) can be used in the following medical contexts, inter alia: Surgical Trocars, Suture, Catheters, Intravenous Lines, Drains, Tubes (e.g. nasogastric, orogastric, endotracheal, tracheal, etc.), Implants (plates, screws, prostheses, hardware, etc.), etc.

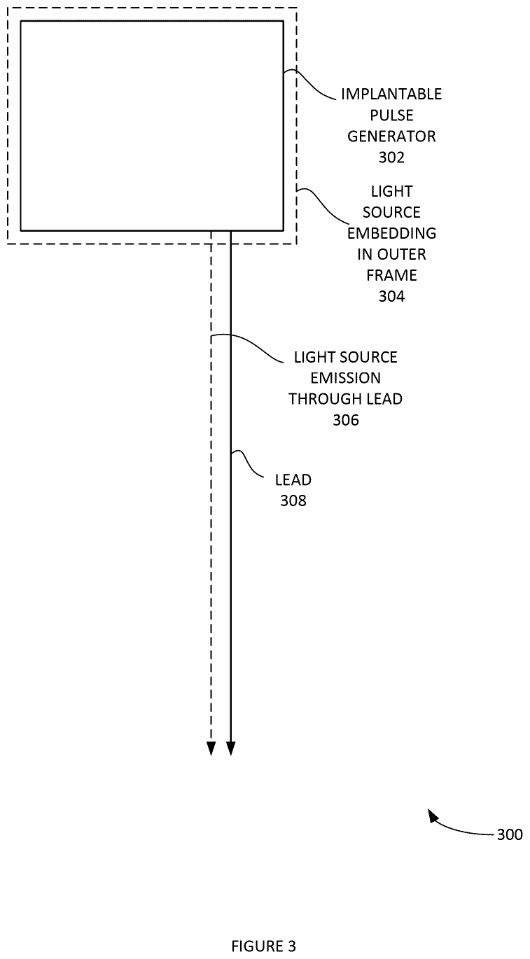

FIG. 3 illustrates an example implantable pulse generator with a light-source embedding in outer frame 300, according to some embodiments.

In some examples, an IPG 302 can be a battery-powered micro-electronic device that is implanted in the body and provide electrical stimulation to the nervous system. IPG 302 can be used for various medical functions (e.g. regulating heart rhythms, managing chronic pain, etc.). IPG 302 can include electric circuits and a battery.

IPG 302 can include Light Source Embedding in Outer Frame 304. Light Source Embedding in Outer Frame 304 can be a germicidal irradiation source (e.g. germicidal irradiation source(s) 104 discussed supra) that emits a germicidal radiation from portions of the surface of IPG 302. For example, Light Source Embedding in Outer Frame 304 can apply UVC-light production.

Lead(s) 308 (e.g. wires) are implanted and provide the electrical stimulation to a specified portion of the nervous system. For example, lead(s) 308 inside veins to carry electrical pulses to the heart, helping it to beat in a specific pattern/frequency. A light source emission through lead 306 can be couples with one or more leads 308. Light source emission through lead 306 can be a germicidal irradiation source (e.g. germicidal irradiation source(s) 104 discussed supra) that emits a germicidal radiation in a locality of lead(s) 308.

It is noted, Light Source Embedding in Outer Frame 304 and/or Light source emission through lead 306 can provide germicidal irradiation of various intensities and periods depending on automated pre-programming and/or external control signals. In one example, IPG 302 can include a wireless communication system (e.g. Wi-Fi, Bluetooth®, etc.). External controls can be used to determine an intensity and/or duration Light Source Embedding in Outer Frame 304 and/or Light source emission through lead 306. Light Source Embedding in Outer Frame 304 and/or Light source emission through lead 306 can operate with separate and controllable intensities and durations. In this way, a medical doctor can externally direct a Light source emission through lead 306 to operate in a specified manner in a specified location (e.g. an artery, etc.), while another Light source emission through lead 306 and Light Source Embedding in Outer Frame 304 are off.

In one example, Lead(s) 308 and/or IPG 302 can include biosensors that detect pathogens. Lead(s) 308 and/or IPG 302 can then cause Light Source Embedding in Outer Frame 304 and/or Light source emission through lead 306 to automatically emit germicidal irradiation of various intensities and periods depending on such factors as, inter alia: the type of pathogen, severity of infection in the site, etc.

In one example, at least one light source (Light Source Embedding in Outer Frame 304 and/or Light source emission through lead 306, etc.) can encapsulate IPG 302. A light source can be built into the frame of the IPG 302 itself. In one example, a type of adhesive capability can be applied to hold a light source directly onto IPG 302 through an adhesive mechanism and/or through a magnetic contact (e.g. and/or an inductive magnetic charging, etc.). In another example, a mesh pocket can hold IPG 302 and one or more light sources.

A lead 308 can be manufactured light source that is built into it. Lead 308 can be connected to IPG 302. In one example, an adhesive capability (e.g. see supra) can be used to apply a light source to a lead insertion.

### CONCLUSION

Although the present embodiments have been described with reference to specific example embodiments, various modifications and changes can be made to these embodiments without departing from the broader spirit and scope of the various embodiments. For example, the various devices, modules, etc. described herein can be enabled and operated using hardware circuitry, firmware, software or any combination of hardware, firmware, and software (e.g. embodied in a machine-readable medium).

In addition, it can be appreciated that the various operations, processes, and methods disclosed herein can be embodied in a machine-readable medium and/or a machine accessible medium compatible with a data processing system (e.g., a computer system), and can be performed in any order (e.g., including using means for achieving the various operations). Accordingly, the specification and drawings are to be regarded in an illustrative rather than a restrictive sense. In some embodiments, the machine-readable medium can be a non-transitory form of machine-readable medium.

What is claimed:

1. An implantable medical device comprising:
a germicidal irradiation source comprising an ultraviolet germicidal irradiation (UVGI) light source, wherein the germicidal irradiation source is on an exterior surface of a portion of implantable medical device, and wherein the UVGI light source is configured to emits a UVGI light to kill or inactivate microorganisms;
an implantable power generator comprising a power source configured to provide power to the implantable medical device and the germicidal irradiation source.

2. The implantable medical device of claim 1, wherein the implantable medical device comprises a pulse generator, a defibrillator, a pacemaker, a cochlear implant, or a ventricular assist device with an external mesh casing.

3. The implantable medical device of claim 1, wherein the wherein the is integrated into an external surface of the pulse generator, the defibrillator, the pacemaker, the cochlear implant, the ventricular assist device, or an artificial heart.

4. The implantable medical device of claim 1, wherein the UVGI light source is configured to provide a disinfection technique employing ultraviolet (UV) light.

5. The implantable medical device of claim 4, wherein the UV light comprises a UV-C light source configured to emit a UV-C light at 180-280 nm.

6. The implantable medical device of claim 5, wherein the implantable power generator comprises a battery-powered micro-electronic device.

7. The implantable medical device of claim 6, wherein the implantable power generator is configured to provide electrical stimulation to a nervous system.

8. The implantable medical device of claim 7, wherein the implantable power generator is configured to regulate a heart rhythm.

9. The implantable medical device of claim 8, wherein the implantable power generator a sensing amplifier.

10. The implantable medical device of claim 9, wherein the sensing amplifier processes an electrical manifestation of series of naturally occurring heart beats as sensed by a heart electrodes.

11. The implantable medical device of claim 10, wherein the implantable medical device comprises an implantable pulse generator.

12. The implantable medical device of claim 11, wherein the germicidal irradiation source comprises a light-source embedding in an outer frame of the implantable pulse generator.

13. The implantable medical device of claim 12, wherein at least one lead is coupled with the implantable pulse generator.

14. The implantable medical device of claim 13, wherein the at least on lead is implanted to a specified portion of the nervous system.

15. The implantable medical device of claim 14, wherein the at least one lead is configured to provide the electrical stimulation to the specified portion of the nervous system.

16. The implantable medical device of claim 15, wherein the at least one lead is configured to carry a series of electrical pulses to the heart.

17. The implantable medical device of claim 16, wherein each lead of the at least one lead comprises a thread-like germicidal irradiation source.

18. The implantable medical device of claim 17, wherein each lead of the at least on lead comprises a biosensor configured detect a pathogen.

19. The implantable medical device of claim 18, wherein each lead causes the germicidal irradiation source and the thread-like germicidal irradiation source to automatically emit a germicidal irradiation of specified intensity and period depending on the type of pathogen the severity of infection as determined by a number the biosensor detection of the pathogen.
